(19) United States
(12) Patent Application Publication
(10) Pub. No.: US 2013/0189244 A1
(43) Pub. Date: Jul. 25, 2013

(54) RECOMBINANT OR TRANSGENIC FACTOR VII COMPOUND HAVING A MAJORITY OF GLYCAN, BIANTENNARY, BISIALYLATED AND NON-FUCOSYLATED FORMS

(71) Applicant: LFB BIOTECHNOLOGIES, Les Ulis (FR)

(72) Inventors: Abdessatar Sami Chtourou, Elancourt (FR); Emmanuel Nony, Antony (FR); Nicolas Bihoreau, Orsay (FR)

(73) Assignee: LFB BIOTECHNOLOGIES, Les Ulis (FR)

(21) Appl. No.: 13/705,948

(22) Filed: Dec. 5, 2012

Related U.S. Application Data
(62) Division of application No. 12/374,269, filed on May 26, 2009, now abandoned.

(30) Foreign Application Priority Data
Aug. 1, 2006 (FR) 0607016

(51) Int. Cl. C12N 9/50

(52) U.S. Cl. CPC C12N 9/50
USPC 424/94.64; 435/219

(57) ABSTRACT

The present invention concerns a recombinant or transgenic factor VII compound, each factor VII molecule of the compound having glycan forms linked to N-glycosylation sites, wherein among all the factor VII molecules in said compound, glycan, biantennary, bisialylated and non-fucosylated forms are in the majority. The invention also concerns such a compound for use as a medication, and a method for preparing said compound, among others.

### CROSS-REFERENCE TO RELATED APPLICATIONS

This application claims priority as a divisional application of U.S. patent application Ser. No. 12/374,269, filed May 26, 2009, which is a §371 application from International Application No. PCT/FR2007/001324, filed Jul. 31, 2007, which claims priority to French Application No. 0607016, filed Aug. 1, 2006. Each of these applications is incorporated herein by reference in its entirety.

### BACKGROUND

Factor VII (FVII) is a vitamin K-dependent glycoprotein which, in activated form (FVIIa), is involved in the coagulation process activating the Factor X and the Factor IX in the presence of calcium and of tissue factor. FVII is secreted in form of a single peptide chain of 406 residues with a molecular weight of about 50 kDa. FVII contains four distinctive structural domains: the N-terminal γ-carboxyl (Gla) domain, two “Epidermal Growth Factor (EGF)-like” domains, and a serine protease domain. The activation of FVII to FVIIa is characterized by the cleavage of the binding Arg₁₅₂-IIe₁₅₃ (Arginine 152-Isoleucine 153). FVIIa consists of a light chain of 152 amino acids with a molecular weight of about 20 kDa and of a heavy chain of 254 amino acids with a molecular weight of about 30 kDa linked by a single disulfide bridge (Cys₁₃₅-Cys₂₆₂). Plasma FVIIa (FVIIa,p) comprises several post-translational modifications: the first ten glutamic acids are γ-carboxylated, Asp₆₃ (aspartic acid) is partially hydroxylated, Ser₅₂ (Serine 52) and Ser₆₀ (Serine 60) are O-glycosylated and carry the Glucose(Xylose)₀₋₂ and Fucose moieties, respectively, Asn₁₄₅ (Asparagine 145) and Asn₃₂₂ (Asparagine 322) are N-glycosylated with mainly biantennary bisialylated complex glycan forms.

FVII is used for the treatment of patients suffering from haemophilia, exhibiting Factor VIII deficiency (type A haemophilia) or Factor IX deficiency (type B haemophilia), and patients exhibiting also further coagulation factors deficiencies, for example a congenital FVII-deficiency. FVII is also recommended for the treatment of cerebro-vascular accidents. It is therefore necessary that FVIIa concentrates for injection are available. The most ancient method for obtaining FVIIa concentrates consisted in the purification of FVIIa from plasma proteins obtained by fractionation.

To this end, the document EP 0 346 241 describes the preparation of a FVIIa-enriched fraction obtained after adsorption, then elution of a fractionation by-product of plasma proteins containing the FVII and the FVIIa and further proteins such as Factors IX (FIX), X (FX) and II (FII), namely the pre-eluate of PPSB (P=prothrombin or FII, P=proconvertin or FVII, S=Stuart Factor or FX and B=antihaemophiliac Factor B or FIX). The disadvantage of this process is that the obtained FVII still contains some traces of other clotting factors.

Likewise, the document EP 0 547 932 describes a manufacturing process of a high purity FVIIa concentrate substantially free of vitamin K-dependent factors and of FVIII. The FVII obtained in this process, despite its purity, exhibits a residual thrombogenic activity.

A major drawback of these processes is that they give only low yields of products.

Moreover, the volume of plasma collected from blood donors remains limited.

Therefore, since the 1980s, the DNA encoding the human Factor VII was isolated (Hagen et al. (1986); Proc. Natl. Acad. Sci. USA; April 83(8):2412-6) and expressed in mammal BHK cells (Baby Hamster Kidney) (document EP 0 200 421). The patent application FR 06 04872 filed by the Applicant also describes the manufacturing of FVIIa in a transgenic animal.

The proteins obtained by these manufacturing methods are made more secure in terms of virus or other pathogenic agents contamination. Furthermore, such processes allow to obtain proteins having a primary sequence, i.e. a chaining between the different amino acids, identical to the primary human sequence. However, the human plasma FVII contains complex post-translational modifications: the first ten glutamic acids are γ-carboxylated, Asp₆₃ is partially hydroxylated (aspartic acid 63), Ser₅₂ (Serine 52) and Ser₆₀ (Serine 60) are O-glycosylated and carry Glucose(Xylose)₀₋₂ and Fucose moieties, respectively, Asn₁₄₅ (Asparagine 145) and Asn₃₂₂ (Asparagine 322) are N-glycosylated mainly with biantennary and bisialylated complex forms. Especially, the addition of N-glycans (glycans linked to asparagine) is particularly important to the correct folding of the protein, the in vitro and in vivo stability, the bioactivity and the pharmacokinetic properties (biodisponibility, for example) of the produced heterologous protein. Thus, variations of all post-translational modifications, or a part thereof, are exposing the protein on one hand, to the risk of being inactive and, on the other hand, to the risk of being immunogenic.

Now, the existing recombinant or transgenic Factors VII can exhibit, owing to their expression in systems different from human systems, a glycosylation which is different from the glycosylation of the human plasma FVII, which can lead to the raise of antibodies directed against the recombinant protein and therefore to a lower efficiency than that of the human FVII purified from human plasma.

Therefore there is a need for therapeutic or prophylactic compositions of FVIIa, the functional properties thereof are near to the human FVII purified from human plasma, and the manufacturing method thereof of compatible with the need of large amounts of this protein.

### SUMMARY

Thus, the invention is related to a composition of recombinant or transgenic Factor VII, each molecule of Factor VII of the composition containing glycan forms bound to N-glycosylation sites, characterized in that among all the molecules of Factor VII of said composition, the majority are biantennary, bisialylated and non fucosylated glycan forms in comparison with all glycan forms bound to N-glycosylation sites of Factor VII of the composition.

Surprisingly, the Applicant discovered that a composition of recombinant or transgenic FVII having a majority of biantennary, bisialylated and non fucosylated forms, exhibits an increased bio-disponibility, a reduced clearance and an increased stability in comparison with a composition of recombinant or transgenic FVII having a lower proportion of bisialylated forms, i.e. in comparison with a composition of recombinant or transgenic FVII having a majority proportion of biantennary, monosialylated and non fucosylated forms.

Therefore, it can be assumed, that the FVII of the invention would be administered to the patient with a lesser frequency and in lower doses in comparison with a composition of recombinant or transgenic FVII having a lower proportion of bisialylated forms, i.e. a majority proportion of monosialylated forms.

Biodisponibility refers to the percentage of administered FVII diffusing into the blood circulation and therefore liable, in particular, to reach the site of hemorrhage.

Clearance refers to the fraction of a completely purified theoretical volume, i.e. no more FVII per time unit is contained. In other words, this corresponds to the hypothetical amount of fluid which will be completely free of the substance in an interval of time unit.

Stability refers to the capacity of FVII to maintain the chemical, physical, microbiological, and biopharmaceutical properties thereof in specific limits during the entire validity thereof.

<<Biantennary, bisialylated and non fucosylated glycan forms>> refer to the forms herebelow:

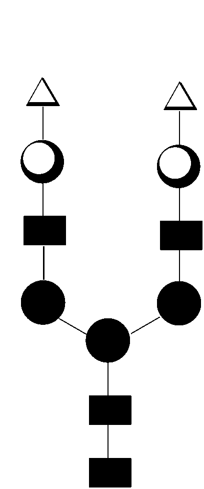

Form A2 (biantennary, bisialylated and non fucosylated)

[symbol] : Sialic acid

[symbol] : Galactose

[symbol] : N-acetylglucosamin (GlcNAc)

[symbol] : Mannose

These glycan forms are bound to N-glycosylation sites consisting of asparagine 145 (Asn145) and asparagine 322 (Asn322). Indeed, the FVII of the invention comprises, as the human FVII, two N-glycosylation sites in positions 145 and 322, and 2 O-glycosylation sites in positions 52 and 60. In a N-glycosylation site, the oligosaccharide chains are linked to an asparagine (N-linked). In a O-glycosylation site, the oligosaccharide chains are linked to a serine. Therefore, each molecule of FVII of the invention comprises two oligosaccharide N-linked chains. However, the molecules of FVII of the composition do not exhibit a homogeneous glycosylation, i.e. all N-linked oligosaccharide chains are not identical. It is a question of a mixture of different glycan forms.

In fact, any FVII, whether plasma, recombinant or transgenic, is present in form of a mixture of several proteins of FVII, these proteins exhibit differences especially in their glycosylation and in differently designated glycoforms. This glycosylation is due to a post-translational processing carried out by cellular organites upon the transfer of FVII protein between the different cellular compartments. This biochemical modification deeply modifies the protein so that the final protein is perfectly structured and thus both active and well tolerated by the organism. This chemical modification contributes to the regulation of the protein activity, and to the localization thereof, as well. Thus, for the whole composition of FVII, and therefore for all the N-linked oligosaccharide chains of the composition, the proportion of each glycan form or of each sugar present in the composition of FVII, can be quantified.

O-glycosylation is not taken into account in the percentage of the different glycans given in the present application.

<<Composition of FVII>> refers to a composition, the only molecular entity of which is the FVII, preferably activated.

Each molecule of FVII of the composition exhibits the same primary sequence but a glycosylation varying from one molecule to another. Thus, <<composition of FVII>> refers to a mixture of molecules having the same primary sequence characterized by its content of glycan forms. For the sake of the invention, expressions <<FVII>> and <<composition of FVII>> are equivalent. Consequently, in the context of the invention, “FVII” refers to a molecule of FVII as such, or to a mixture of FVII molecules having the above mentioned characteristics.

The composition of FVII of the invention is a composition of FVII containing mainly biantennary, bisialylated and non fucosylated glycan forms. This means that among all the N-linked oligosaccharides of the composition, i.e. all the glycan forms bound to N-glycosylation sites of Factor VII, the biantennary, bisialylated and non fucosylated forms are the most represented.

Advantageously, the proportion of biantennary, bisialylated and non fucosylated glycan forms is higher than or equal to 30%, 40%, 50%, 60%, 70%, 80%, 90% or yet 95%. In a particularly advantageous way, the proportion of biantennary, bisialylated and non fucosylated glycan forms is higher than or equal to 45%. In a particularly advantageous way, the proportion of biantennary, bisialylated and non fucosylated glycan forms is comprised between 45% and 65%, and preferentially, comprised between 50% and 60%.

The rates of sialylated species can be empirically determined by HPCE-LIF analysis (High Performance Capillary Electrophoresis-Laser Induced Fluorescence) and/or NP-HPLC (Normal Phase High Performance Liquid Chromatography) with quantification by measuring the area of the peaks corresponding to different glycans, or by any method known to persons skilled in the art.

The composition of FVII of the invention can also comprise minor biantennary, monosialylated, and triantennary forms, and also neutral forms not exhibiting sialic acids.

<<Recombinant or transgenic FVII>> refers to any FVII resulting from genetic engineering, i.e. produced by cells the DNA of which was modified by genetic recombination so that they express a molecule of FVII, and exhibit the described glycosylation features.

Thus, the FVII of the invention results from the transcription, then the translation of a DNA molecule encoding the FVII in a cellular host or in a transgenic animal. The recombinant or transgenic FVII of the invention can be obtained by standard techniques known to persons skilled in the art, allowing the expression of a protein in a biological system.

More particularly, <<recombinant VII>> refers to any FVII obtained by genetic recombination and expressed in a cultured cell line. By the way of example, the following cell lines can be mentioned: BHK (Baby Hamster Kidney) and namely BHK tk⁻ts13 (CRL 10314, Waechter and Baserga, Proc. Natl. Acad. Sci. USA 79:1106-1110. 1982), CHO (ATCC CCL 61), COS-1 (ATCC CRL 1650), HEK293 (ATCC CRL 1573; Graham et al., J. Gen. Virol. 36:59-72, 1977), Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1) and DUKX cells (CHO cell line) (Urlaub and Chasin, Proc. Natl. Acad. Sci. USA 77:4216-4220, 1980), 3T3 cells, Namalwa cells, or BHK cells adapted to serum-free culture (Document U.S. Pat. No. 6,903,069).

Furthermore, more particularly <<transgenic FVII<< refers to any FVII obtained by genetic recombination and expressed in a living tissue, in an animal or in a plant.

The proportion of bisialylated forms of the invention can be obtained in different ways.

### DESCRIPTION OF THE DRAWINGS

FIG. 1: Extraction and purification of the composition of FVII obtained in Example 1.

FIG. 2: Deconvoluted mass spectra ESI of peptides carrying N-glycosylation sites.

FIG. 3: Electropherograms HPCE-LIF after deglycosylation of the FVII by the PNGase F;

Legend: Electropherogram top: FVIIa,p; both electropherograms center: FVII-Tg; electropherogram bottom: FVIIa,r.

FIG. 4: Characterization of FVII by NP-HPLC;

Legend: Chromatogram top: FVIIa,p; chromatogram center: FVII-Tg; chromatogram bottom: FVIIa,r.

FIG. 5: Identification of the majority glycan forms of FVII-Tg by MALDI-TOFMS.

FIG. 6: Identification of the majority glycan forms of FVIIa,r by MALDI-TOFMS.

FIG. 7: HPCE-LIF Analyses of the resialylation in vitro : (bottom) oligosaccharidic map of the native FVII-Tg; (top) oligosaccharidic map of the FVII-Tg after resialylation.

FIG. 8: Kinetics of sialylation of FVII-Tg according to the percentage of biantennary, bisialylated, non fucosylated (A2) and fucosylated (A2F) forms in time.

FIG. 9: Results of the preliminary PK (PK: pharmocokinetics) comparative study in rabbit, transgenic non resialylated FVII (FVIITgNRS) compared to the transgenic resialylated FVII (FVIITgRS): semilog curves of elimination.

### DESCRIPTION

In a particular embodiment, the FVII of the invention is expressed in a microorganism, in a cell, in a plant or in an animal imparting the described glycosylation features, i.e. in majority biantennary, bisialylated and non fucosylated forms.

In a further embodiment, the FVII of the invention is expressed in a microorganism, in a plant or in an animal not allowing to obtain a composition of FVII exhibiting mainly biantennary bisialylated and non fucosylated forms, the sialylation being carried out subsequently in vitro using one or more enzymes in order to carry out the desired sialylation, i.e. the biantennary and bisialylated forms become majority and the triantennary forms become trisialylated.

By way of example, a sialyltransferase can be made to act, in vitro, on a composition of FVII selected for its favourable properties, under suitable conditions, in order to allow the desired sialylation. Thus, the composition of FVII of the invention is liable to be obtained by the action of a sialyltransferase on a partially sialylated composition of FVII (starting composition of FVII). Advantageously, the starting composition of FVII exhibits in majority biantennary, monosialylated glycan forms. Advantageously, the starting composition of FVII exhibits a majority of biantennary, monosialylated and non fucosylated glycan forms. The action of the sialyltransferase allows to graft an additional sialic acid on the monosialylated forms converting them to a bisialylated form. Advantageously, these biantennary, monosialylated forms are present in the starting composition of FVII at a proportion higher than 40%, in a particularly advantageous way at a proportion higher than 50%, or yet 60%. Advantageously, the starting composition exhibits a proportion of biantennary, monosialylated and non fucosylated glycan forms higher than 20%, or particularly higher than 30%, than 40%, or yet 50%.

Advantageously, at least some of the sialic acids of the starting composition of FVII imply α2-6-links. In a particularly advantageous way, the proportion of sialic acids implying α2-6-links is higher than 60%, or yet higher than 70%, 80%, or 90%. Particularly, this proportion is comprised between 60% and 90%.

Preferentially, all sialic acids of the starting composition of FVII imply α2-6-links.

In a particular embodiment, if the starting composition of FVII contains a too high proportion of fucosylated forms, for example higher than 50%, or yet higher than 60%, it is possible to obtain biantennary, bisialylated and non fucosylated forms using one or more enzymes allowing to defucosylate the composition. The use of a fucosidase can be mentioned by way of example, for a period of time necessary for obtaining a majority of biantennary, bisialylated and non fucosylated glycan forms.

In a particularly advantageous way, the starting composition of FVII is selected for its low immunogenicity.

Advantageously, the starting composition is the composition of FVII described in the document FR 06 04872 the content of which is considered as included in the present document. Advantageously, the FVII of the invention is a polypeptide, the peptide sequence thereof can be that of the natural human FVII, i.e. the sequence present in man exhibiting no problems associated to FVII. Such a sequence can be encoded for example by the sequence 1b described in the document EP 0 200 421.

Advantageously, the sequence of FVII of the invention is the sequence of SEQ ID NO: 1.

In a further embodiment, the FVII of the invention can be a variant of the natural human FVII, as far as this variant is not more immunogenic than the natural FVII. Thus the peptide sequence of this variant can exhibit an identity of at least 70%, and advantageously of at least 80% or 90%, and in yet more advantageously, an identity of at least 99% with the sequence of the natural human FVII, such a variant having substantially the same biological activity as the natural FVII.

Moreover, the FVII of the invention refers also to any sequence of FVII modified so that the biological activity of the protein is reduced by comparison with the natural human FVII. The recombinant inactivated human FVII, FFR-FVIIa, used for the treatment or prophylaxy of thromboses (Hoist et al., Eur. J. Vasc. Endovasc. Surg., 1998 June, 15(6): 515-520), can be mentioned by the way of example. Such FVII are polypeptides exhibiting an amino acid sequence which differs from the sequence of the natural FVII by insertion, deletion or substitution of one or more amino acids.

The biological activity of FVII of the invention can be quantified by measuring the capacity of a composition of FVII to induce the blood coagulation by use of a FVII-deficient plasma and of thromboplastin, as for example described in the U.S. Pat. No. 5,997,864. In the test described in the patent U.S. Pat. No. 5,997,864, the biological activity is expressed by a reduction of the coagulation time compared to a control sample, and is converted to <<units of FVII>> in comparison with a standard of human serum (pool) containing 1 unit (1 U of FVII activity)/ml of serum.

The composition of FVII of the invention exhibits features of glycosylation nearing those of the plasma FVII. In fact, the major N-glycan form of plasma FVII (or composition of plasma FVII) is also the biantennary, bisialylated form.

Advantageously, the proportion of biantennary, bisialylated (fucosylated and non fucosylated) forms of FVII of the composition of the invention is higher than 30%, or 40%, or 50%. In a particularly advantageous way, the proportion of biantennary, bisialylated forms is higher than 60%, or 70%, or 80% or yet 90%. In a particularly advantageous way, the proportion of bisialylated (fucosylated and non fucosylated) forms is comprised between 50% and 80% or between 60% and 90%, or preferentially, between 70% and 85%.

Advantageously, the proportion of fucose of the composition of FVII of the invention is higher than 20%, and is advantageously comprised between 20% and 50%. This proportion corresponds to the proportion of fucose measured for all glycan forms of FVII of the composition.

This feature is one among the advantages of the FVII of the invention. Indeed, the commercially available recombinant FVII exhibits a 100% proportion of fucosylation, while the plasma FVII has a proportion of fucosylation about 16%. Thus, the fucosylation of FVII of the invention is near to that of plasma FVII, what gives an advantage to the FVII of the invention in terms of innocuity.

Advantageously, at least some of sialic acids of the composition of Factor VII of the invention imply α2-6-links. In a particularly advantageous way, the proportion of sialic acids implying α2-6-links is higher than 60%, or yet higher than 70%, 80%, or 90%. Particularly, this proportion is comprised between 60% and 90%.

Thus, the composition of FVII of the invention comprises a non zero proportion of sialic acid implying α2-6-links. This is an advantage over the recombinant commercial FVII comprising only sialic acids implying α2-3-links, while these are contained in the plasma FVII.

In a particularly preferred embodiment of the invention, all sialic acids of the composition of FVII of the invention imply α2-6-links.

In a particularly preferred way, all sialic acids imply α2,6-links, i.e. all sialic acids are bound to galactose by a α2,6-link, and, in particular, at least 90% of sialic acids of FVII imply α2,6-links. The composition of FVII according to the invention can moreover comprise sialic acids implying α2-3-links.

The fact that the sialic acids of FVII of the composition imply α2,6-branchings is one among the advantages of the FVII of the invention. Indeed, the sialic acids of commercially available recombinant FVII imply only α2,3-links. The plasma FVII is a mixture of these two isomers. Such a plasma FVII contains for example 40% of isomers α2,3 and 60% of isomers α2,6. However, the latter comprises more α2,6-links, what brings the FVII of the invention nearer to the plasma FVII.

In a further embodiment, some sialic acids of the composition of FVII of the invention imply α2-3-links.

Thus, in a particular embodiment of the invention, the recombinant or transgenic FVII of the composition exhibits mainly biantennary, bisialylated and non fucosylated glycan forms compared to all the glycan forms bound to N-glycosylation sites of Factor VII, and a proportion of sialic acids implying α2-6-links higher than 90%.

In a particularly preferred embodiment of the invention, the recombinant or transgenic FVII of the composition exhibits mainly biantennary, bisialylated and non fucosylated glycan forms compared to all the glycan forms bound to N-glycosylation sites of Factor VII, and a proportion of sialic acids implying α2-6-links equal to 100%.

In a particular embodiment of the invention, the recombinant or transgenic FVII of the composition exhibits mainly biantennary, bisialylated and non fucosylated glycan forms compared to all glycan forms bound to N-glycosylation sites of Factor VII, the proportion of fucose of the composition of FVII being comprised between 20% and 50%.

In a particular embodiment of the invention, the recombinant or transgenic FVII of the composition exhibits in majority biantennary, bisialylated and non fucosylated glycan forms compared to all the glycan forms bound to N-glycosylation sites of Factor VII, all sialic acids implying α2-6-links, and the proportion of fucose of the composition of FVII being comprised between 20% and 50%.

Advantageously, the composition of FVII of the invention is liable to be produced by a non human, transgenic mammal.

In this embodiment, the composition of FVII of the invention will therefore be considered <<transgenic>>. Transgenic mammal refers to any mammal except of human being, genetically manipulated in order to express an exogenous protein, for example rabbit, goat, mouse, rat, bovine, horse, pig, insects, sheep, this list being not limitative. The exogenous protein is the FVII, preferably the human FVII. The non human transgenic mammal can, in addition to the FVII, express an exogenous enzyme so as to impart the desired sialylation to the composition of transgenic FVII. On this account, the non human transgenic animal can co-express the gene encoding the FVII and the gene encoding a sialyltransferase.

In a particular embodiment of the invention, the transgenic FVII of the invention is expressed in the mammary glands of the transgenic mammal and produced in the milk thereof. On this account, the expression of the transgene is carried out in a tissue-dependent way by means of a promoter ensuring the production of the transgene in the mammary glands of the animal. The WAP promoter (whey acidic protein), the casein promoter, in particular the β-casein or α-casein promoter, the β-lactoglobulin promoter, the α-lactalbumin promoter can be cited, this list being not limitative.

Advantageously, the composition of FVII of the invention is liable to be produced by a transgenic female rabbit, said composition being further subjected to a sialylation in vitro so that the majority will be biantennary, bisialylated forms.

The rabbit is a particularly advantageous species for the production of therapeutic protein, as the rabbit appears to be insensitive to prions, especially to transmissible spongiform sub-acute encephalopathy, which is a major public health issue.

Furthermore, the species barrier between rabbit and man is important. Conversely, the species barrier between man and hamster, which is the biological system where the commercially available recombinant FVII is produced, is less important.

Thus, the production of FVII in rabbit is advantageous in terms of safety against the transmission of pathogenic agents, including non conventional pathogenic agents of prions type. In a preferred embodiment of the invention, the FVII of the invention is produced in the mammary glands of transgenic female rabbits.

The secretion of the protein of interest by mammary glands, allowing the secretion into the milk of a transgenic mammal, is a technique well known to persons skilled in the art implying the control of the expression of the recombinant protein in a tissue-dependent manner. The tissue control of the expression is carried out thanks to sequences allowing the protein expression to be oriented towards a particular tissue of the animal. These sequences are namely promoter sequences and signal peptide sequences, as well.

Examples of promoters driving the expression of a protein of interest in the mammary glands are the WAP promoter (whey acidic protein), the casein promoter, especially the β-casein, the α-casein promoter, the β-lactoglobulin, the α-lactalbumin promoter, this list is not limitative. In a particularly advantageous manner, the expression in the mammary glands of the female rabbit is performed under the β-casein promoter control.

A production method of a recombinant protein in the milk of a transgenic animal can include the following steps: a synthetic DNA molecule comprising a gene encoding the human FVII, this gene, being under the control of a promoter of a naturally secreted protein into the milk, is integrated into the embryo of a non-human mammal. The embryo is subsequently placed into a female mammal of the same species. Once the mammal obtained from the embryo is sufficiently developed, the lactation of the mammal is induced, next the milk is collected. Then the milk contains the transgenic FVII of interest.

An example of a process for preparing transgenic protein in the milk of a female mammal other than man is described in the document EP 0 264 166, the teaching of which can be referred to for the production of the FVII of the invention.

A further example of a process for preparing a protein in the milk of a mammal female other than man is given in the document EP 0 527 063, the teaching of which can be referred to for the production of the FVII of the invention.

The composition of FVII produced in the mammary glands of female rabbit is characterized in that at least some of the sialic acids of Factor VII imply α2-6-links.

In a particularly preferred way, all the sialic acids imply α2,6-links, and in particular, at least 90% of sialic acids of FVII imply α2,6-links. Moreover the composition of FVII according to the invention can contain sialic acids of α2-3-links.

In a particularly advantageous manner, the proportion of sialic acids implying α2-6-links is higher than 60%, or yet higher than 70%, 80%, or 90%. In particular, this proportion is comprised between 60% and 90%.

Among the biantennary, monosialylated glycan forms of the composition of FVII expressed in the female rabbit, the majority glycan forms are non fucosylated. Advantageously, these biantennary, monosialylated and non fucosylated glycan forms are present in the FVII of this composition at a proportion higher than 20%. Advantageously, this proportion is higher than 25%, or yet higher than 40%.

In an embodiment of the invention, the proportion of fucosylation of FVII of this composition of the invention is comprised between 20% and 50%. In a further embodiment of the invention, this proportion can be lower than 15%.

The transgenic FVII from female rabbit comprises several post-translational modifications: the first nine or ten N-terminal glutamic acids are γ-carboxylated, Asp₆₃ (Asparagine63) is partially hydroxylated, Ser₅₂ (Serine 52) and Ser₆₀ (Serine 60) are O-glycosylated and carry Glucose(Xylose)₀₋₂ and Fucose moieties, respectively, Asn₁₄₅ and Asn₃₂₂ are N-glycosylated mainly by biantennary monosialylated glycan complex forms.

Such a composition of FVII produced in the mammary glands of female rabbit is described in the document FR 06 04872, the content of which is incorporated herein into the present teaching.

The FVII produced in the milk by transgenic mammals can be purified from the milk by use of techniques known to persons skilled in the art.

For example, a purification method of the protein of interest from milk such as described in the patent U.S. Pat. No. 6,268,487, can include the following steps consisting of: a) subjecting the milk to a tangential filtration through a membrane having a sufficient porosity for forming a retentate and a permeate, the permeate contains the exogenous protein, b) subjecting the permeate to a capture apparatus by chromatography in a way to displace the exogenous protein and to obtain an effluent, c) combining the effluent and the retentate, d) repeating the steps a) to c) until the separation of FVII from the lipids, the casein micelles, and that the FVII should be recovered at least to 75%.

A further purification technique of the FVII produced in the milk of a transgenic mammal is described in the patent application FR 06 04864 filed by the Applicant, the content of which is incorporated by reference. This extraction and purification process of FVII (Process A), contained in the milk of a transgenic animal, comprises the following steps of:

a) extracting the FVII from the milk, the Factor VII being bound to organic and/or inorganic salts and/or complexes of calcium of said milk, by precipitation of calcium compounds obtained by addition of a soluble salt to the milk, the anion thereof is selected for its capability to form said insoluble calcium compounds in order to release in this way the Factor VII from said salts and/or complexes, the Factor VII being present in a liquid phase,

b) separating the protein-enriched liquid phase from the precipitate of calcium compounds, said liquid phase being further separated in a lipidic phase and in an aqueous non lipidic phase containing the protein,

c) subjecting the aqueous non lipidic phase to an affinity chromatography step, using an elution buffer based on a phosphate salt in a predetermined concentration, and

d) subjecting the eluate of Factor VII obtained according to the step c), to two or three chromatography steps on anion exchange columns of weak base type using buffers suitable for successive elutions of the Factor VII retained on said columns. Indeed, the Applicant has surprisingly noticed that the FVII, even if placed under the control of a promoter of a protein naturally produced in the lactoserum, such as the WAP promoter or the β-casein promoter for example, is nevertheless liable to be associated with the calcium ions of the milk, and thus with the casein micelles.

A further technique of purification of FVII produced in the milk of a transgenic mammal is described in the patent application FR 06 11536 filed by the Applicant, the content thereof being incorporated by reference. This process of extraction and of purification of FVII contained in the milk of a transgenic animal (Process B), comprising steps of:

a) skimming and delipidation of said milk,

b) passage of the delipidated and skimmed fraction containing the said protein on a chromatographic support with a grafted ligand exhibiting both hydrophobic and ionic character, under pH conditions allowing that the said protein be retained on said support,

d) elution of the protein,

e) purification of the eluted fraction by removal of milk proteins from said eluted fractions, and

e) recovery of said protein.

When the composition of FVII is produced by a transgenic female rabbit, it is subjected to a sialylation in vitro so that the biantennary, bisialylated forms will be majority.

In a particular embodiment of the invention, the sialylation is performed by use of a sialyl-transferase, for example the α2,6-(N)-sialyl-transferase (or β-D-galactosyl-β1,4-N-acetyl-β-D-glucosamin-α2,6-sialyltransferase), or the Gal beta 1,3GalNAc alpha 2,3-sialyltransferase, or the Gal beta 1,3(4) GlcNAc alpha 2,3 sialyltransferase, or GalNAc alpha-2,6-sialyltransferase I, these enzymes being commercially available.

Preferentially, the used sialyltransferase is a sialyltransferase allowing to transfer sialic acids via a α2,6-link. Indeed, it is advantageous that the composition of FVII of the invention exhibits sialic acids implying α2-6-links, because this isomer is more represented in the plasma FVII.

The sialylation can be performed with a sialic acid donor substrate, as for example sialic acid as such or any molecule comprising one or more acid sialic groups and which is liable to release sialic acid groups.

According to an embodiment of the invention, if the enzyme is α2,6-(N)-sialyltransferase, the substrate is the cytidine-5′-monophospho-N-acetyl-neuraminic acid, in a reaction medium suitable for the transfer of the sialic acid from the sialic acid donor group to the FVII, the biantennary, bisialylated forms becoming majority. This reaction medium can be based for example on a buffer consisting of morpholino-3-propanesulfonic acid, and a buffer based, for example, on Tween.

According to a further embodiment of the invention, the substrate can be synthesized in the reaction medium, including in this medium a cytidine monophosphate (CMP)-sialic acid synthetase, sialic acid, CTP (cytidine triphosphate) and a sufficient amount of a divalent metal cation in order to allow that the reaction takes place. By way of example, the divalent metal cation can be the calcium ion, the zinc ion, the magnesium ion, the chromium ion, the copper ion, the iron ion or the cobalt ion.

Whatever the method applied to carry out the sialylation of the composition of FVII, the reaction is always carried out for a sufficient period of time and under suitable conditions allowing a sufficient increase in bisialylated forms, so that they become majority. For information only, the reaction can be carried out for at least 0.5 hours, and, more especially, at least 5 hours, in a particularly advantageous way for 7 hours, or yet for 8 hours, 9 hours, even 10 hours. Preferentially, the incubation takes place over night. In particular, this reaction will be performed for periods of time comprised between 5 and 12 hours.

Advantageously, the FVII of the composition of invention is activated (FVIIa).

On this account, the FVIIa can exhibit a coagulation activity 25 to 100 times higher than the FVII (non activated), upon the interaction of the latter with the tissue factor (TF) for and on behalf of the former. The activation of the FVII results, in vivo, from the cleavage of the zymogen by different proteases (FIXa, FXa, FVIIa) in two chains linked by a disulfide bridge. FVIIa alone exhibits a very poor enzyme activity, but in complex with its cofactor, the tissue factor (TF), triggers the coagulation process by activating the FX and the FIX. The FVIIa is the coagulation factor responsible for haemostasis in haemophiliacs with circulating antibodies, for example. In a particularly advantageous manner, the FVII of the invention is completely activated. Advantageously, the FVIIa of the invention comprises several post-translational modifications: the first nine or ten N-terminal glutamic acids are γ-carboxylated, Asp₆₃ is partially hydroxylated, Ser₅₂ and Ser₆₀ are O-glycosylated and carry Glucose(Xylose)₀₋₂ and Fucose moieties, respectively, Asn₁₄₅ and Asn₃₂₂ are N-glycosylated mainly with complex biantennary, bisialylated and non fucosylated forms.

The activation of the FVII can also result from a process carried out in vitro, for example upon the purification of FVII of the invention (see Example 2).

Thus, the FVIIa of the invention is constituted of a light chain of 152 amino acids with a molecular weight of about 20 kDa and of a heavy chain of 254 amino acids with a molecular weight of about 30 kDa linked one to another by a single disulfide bridge (Cys₁₃₅-Cys₂₆₂).

Thus the FVII of the invention is an activated FVII having an activity and a structure near to the plasma FVII.

FVIIa exhibits a clotting activity 25 to 100 times higher than the FVII upon interaction with the tissue factor (TF).

In an embodiment of the invention, the FVII can be activated in vitro by Factors Xa, VIIa, IIa, IXa and XIIa.

The FVII of the invention can also be activated upon the purification process thereof.

A further object of the invention is a composition of FVII of the invention to be used as medicament.

A further object of the invention is the use of a composition of Factor VII according to the invention, for preparing a medicament for the treatment of patients suffering from heamophilia. A further object of the invention is the use of a composition of Factor VII according to the invention for preparing a medicament intended for the treatment of multiple hemorrhagic traumas.

A further object of the invention is the use of a composition of Factor VII according to the invention for preparing a medicament intended for the treatment of bleedings due to an overdose of anticoagulants.

A further object of the invention is a pharmaceutical composition comprising the Factor VII according to the invention and an excipient and/or a pharmaceutically acceptable carrier.

A further object of the invention is a process for preparing a composition of recombinant or transgenic Factor VII, each molecule of Factor VII of the composition comprising glycan forms bound to N-glycosylation sites, and among all the molecules of Factor VII of said composition, the biantennary, bisialylated glycan forms are majority, comprising a step of sialylation by contacting a composition of transgenic or recombinant Factor VII partially sialylated as defined hereabove with a sialic acid donor substrate and a sialyltransferase, in a suitable reaction medium in order to allow the activity of the sialyltransferase, for a sufficient period of time and under suitable conditions to allow the transfer of the sialic acid from the sialic acid donor substrate to FVII and a sufficient increase in bisialylated forms so that the said bisialylated forms become majority. Conditions to carry out the reaction are described hereabove, and in the examples, as well.

<<Partially sialylated>> refers to a composition of FVII the glycan forms of which bound to N are not all bisialylated, i.e. some forms are monosialylated. Advantageously, these biantennary, monosialylated forms are present at a proportion higher than 40%, in a particularly advantageous way higher than 50%, or yet 60%. Advantageously, the proportion of biantennary, monosialylated and non fucosylated glycan forms is higher than 20%, or in a particularly higher than 30%, than 40%, or yet than 50%.

Advantageously, the sialyltransferase is α2,6-(N)-sialyltransferase (or β-D-Galactosyl-β1,4-N-acetyl-β-D-glucosamine-α2,6-sialyltransferase), or Gal beta 1,3GalNAc alpha 2,3-sialyltransferase, or Gal beta 1,3(4) GlcNAc alpha 2,3 sialyltransferase, or GalNAc alpha-2,6-sialyltransferase I.

Preferentially, the used sialyltransferase is a sialyltransferase allowing the transfer of sialic acids via a α2,6-link. Indeed, it is an advantage that the FVII of the composition of the invention exhibits sialic acids implying α2-6-links, because this isomer is more present in the plasma FVII.

The sialylation can be performed with any sialic acid donor substrate.

According to an embodiment, if the enzyme is the α2,6-(N)-sialyltransferase, the substrate is the cytidine-5′-monophospho-N-acetylneuraminic acid, in a suitable reaction medium to allow the transfer of the sialic acid from the sialic acid donor group to FVII, the biantennary, bisialylated forms becoming majority.

The reaction medium can be based on a tenside mixture biologically compatible, such as Tween®80 or Triton®X-100 or a mixture thereof in a concentration from 0.01% to 0.2%, or a divalent metal cation, such as the cations Ca²⁺, Mn²⁺, Mg²⁺ or Co²⁺, Ca²⁺ being preferred, in a concentration comprised between 5 mM and 10 mM. This reaction medium can further include ionic strength adjustment agents and/or agents maintaining the pH of the medium, such as sodium cacodylate, morpholino-3-propanesulfonic acid, Tris and NaCl in varying concentration from 40 mM to 60 mM. The pH values are typically comprised between 6 and 7.5. The reaction medium can further comprise BSA (Bovine Serum Albumin) at a concentration ranging from 0.05 and 0.15 mg/ml.

According to a further embodiment, the substrate can be synthesized in the reaction medium by introduction into this medium of a CMP-sialic acid synthetase, sialic acid, CTP (cytidine triphosphate), and of a sufficient amount of a divalent metal cation, examples thereof are mentioned above.

Whatever the method of sialylation of the composition of FVII, the reaction is always carried out for a sufficient period of time and under suitable conditions in order to allow a sufficient increase in bisialylated forms so that they become majority, as defined herebove.

When the process uses an immobilized enzyme, the reaction time is preferably comprised between 0.5 to 3 hours, at a temperature advantageously comprised between 4 and 37° C., preferably between 4° C. and 20° C.

When the process is carried out in a batch reaction, the reaction time is preferably comprised between 1 and 9 hours, preferably between 1 and 6 hours, at a temperature advantageously comprised between 4 and 37° C., preferably between 4° C. and 20° C.

Preferably, the process of the invention is a process aiming to improve the biodisponibility of the composition of partially sialylated transgenic or recombinant Factor VII.

This improvement in the biodisposibility is obtained by contacting said composition with a sialic acid donor substrate and a sialyltransferase, such as set forth hereabove.

<<Improving the biodisponibility>> refers to an increase of at least 5%, or of at least 10%, or advantageously of at least 30% or 50%, and in a preferential way, of at least 80% or 90% of the biodisponibility of the composition of FVII compared to the same composition of FVII the sialylation thereof was not modified.

In a further particular embodiment, prior to the sialylation step, a step of galactosylation is carried out. This step aims to graft a galactose on galactose-deficient forms, i.e. the agalactosylated and monogalactosylated forms of FVII. Galactose is fixed to the GlcNAc, and will be liable to fix a sialic acid residue in the subsequent sialylation step. This galactosylation step can be carried out by use of a galactosyl-transferase, in a reaction medium including UDP-gal uridine (5′-diphosphogalactose), known to persons skilled in the art.

Advantageously, the majority glycan forms of partially sialylated FVII composition are of a complex biantennary, monosialylated type.

Such glycan forms are depicted herebelow:

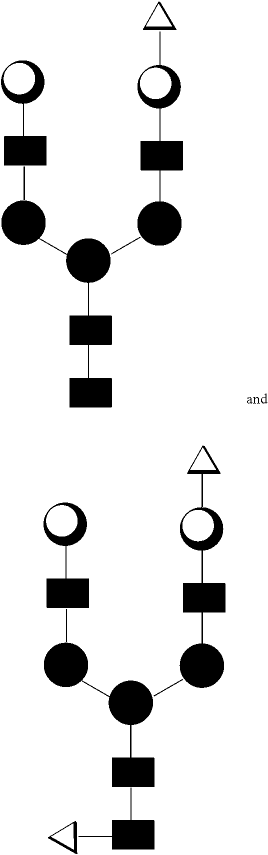

[symbol] : Sialic acid

[symbol] : Galactose

[symbol] : N-acetylglucosamine (GlcNAc)

[symbol] : Mannose

[symbol] : Fucose

In a particular embodiment of the invention, the composition of partially sialylated FVII comprises also biantennary non sialylated (fucosylated or non fucosylated), triantennary non sialylated (fucosylated or non fucosylated), and bisialylated (fucosylated or non fucosylated) complex forms.

Advantageously, among the biantennary, monosialylated glycan forms of the said composition of partially sialylated FVII, the majority glycan forms are non fucosylated.

Advantageously, the composition of partially sialylated FVII exhibits at least some of the sialic acids implying α2-6-links, as previously mentioned.

Preferably, the process comprises further, prior to the sialylation step, a step of production of the composition of partially sialylated transgenic FVII by transgenic female rabbits. This step is carried out as previously described. This step can also be carried out prior to the step of galactosylation.

Advantageously, the FVII of the composition of partially sialylated FVII is activated.

The process of the invention allows to obtain among all the molecules of Factor VII of said composition a majority proportion of biantennary, bisialylated forms.

Advantageously, the sialic acid donor group is the cytidine-5′-monophospho-N-acetylneuraminic acid and the sialyltransferase is the α2,6-(N)-sialyl-transferase.

Such a composition of partially sialylated FVII can be a composition of transgenic FVII produced in the mammary glands of a transgenic female rabbit.

In a particularly advantageous way, the composition of partially sialylated FVII is the composition described in the document FR 06 04872, the content of which is considered as included in the present document.

Further aspects and advantages of the invention will be described in the following Examples, which are given only by way of illustration of the invention, of which they do not constitute in any way a limitation thereof.

### Abbreviations

FVII-Tg=FVIIa-Tg: activated transgenic FVII according to the invention

FVII-r=FVIIa-r: commercially available recombinant activated FVII

FVII-p=FVIIa-p: activated FVII of plasma origin, i.e. purified from human plasma.

MALDI-TOF: Matrix Assisted Laser Desorption Ionisation—Time of Flight

HPCE-LIF: High Performance Capillary Electrophoresis-Laser Induced Fluorescence

ESI-MS: Mass spectrometry-ionisation <<Electrospray>>.

LC-ESIMS: Liquid chromatography-Mass spectrometry-ionisation <<Electrospray>>

NP-HPLC: Normal Phase High Performance Liquid Chromatography

PNGase F: Peptide: N-glycosidase F

LC-MS: Liquid Chromatography-Mass Spectrometry

### EXAMPLES

### Example 1

### Production of Transgenic Female Rabbits Producing a Protein of Human FVII in Their Milk

First, a plasmid p1 is prepared by introduction of the sequence of the WAP gene (described by Devinoy et al., Nucleic Acids Research, vol. 16, no. 16, 25 août 1988, p. 8180) into the polylinker of the vector p-poly III-I (described in the document Lathe et al., Gene (1987) 57, 193-201).

The plasmid p2, obtained from the plasmid p1, contains the promoter of the WAP gene of rabbit and the gene of human FVII.

The transgenic female rabbits were obtained by the classical technique of microinjection (Brinster et al., Proc. Natl. Acad. Sci. USA (1985) 82, 4438-4442). 1-2 pl containing 500 copies of the gene were injected into the male pronucleus of rabbit embryos. The fragments of this vector containing the recombined genes were microinjected. Subsequently, the embryos were transferred into the oviduct of hormonally prepared adoptive females. About 10% of the manipulated embryos gave birth to young rabbits and 2-5% of the manipulated embryos to transgenic young rabbits. The presence of transgenes was revealed by the technique of transfer of Southern from DNA extracted from rabbit tails. The concentrations of FVII in the blood and in the milk of the animals were assessed by specific radioimmunological assays.

The biological activity of FVII was assessed by addition of milk to the cell culture medium or to the rabbit mammary explants culture medium.

### Example 2

### Extraction and Purification of the Obtained FVII

a) Extraction of FVII

500 ml of raw, unskimmed milk, diluted with 9 volumes of 0.25 M sodium phosphate, pH 8.2, were used. After stirring for 30 minutes at room temperature, the aqueous FVII-enriched phase is subjected to a centrifugation at 10000g for 1 hour at 15° C. (centrifuge Sorval Evolution RC—6700 rev/min—rotor SLC-6000). 6 pots of about 835 ml are necessary.

After centrifugation, three phases are present: a lipidic phase on the surface (cream), an aqueous non lipidic clear FVII-enriched phase (majority phase) and a white solid phase in the residue (precipitates of insoluble caseins and of calcium compounds).

The aqueous FVII-enriched non lipidic phase is collected with a peristaltic pump up to the cream phase. The cream phase is collected separately. The solid phase (precipitate) is discarded.

The non lipidic aqueous phase, however, still comprising very low amounts of lipids, is filtered through a sequence of filters (Pall SLK7002U010ZP—glass fibers prefilter with a pore size of 1 μm—then Pall SLK7002NXP—Nylon 66 with a pore size of 0.45 μm). At the end of the filtration, the lipidic phase is passed on this filtration sequence which retains completely the fat globules of the milk, and the filtrate is clear.

The filtered non lipidic aqueous phase is then dialyzed on an ultrafiltration membrane (Millipore Biomax 50 kDa-0.1 m²) to make it compatible with the chromatographic phase. The FVII with a molecular weight of about 50 kDa does not filter through the membrane, unlike the salts, the sugars and the peptides of the milk. In a first time, the solution (about 5 000 ml) is concentrated to 500 ml, then a dialysis by ultrafiltration, maintaining the constant volume, allows to remove the electrolytes and to prepare the biological material for the chromatographic step. The dialysis buffer is 0.025M sodium phosphate, pH 8.2.

This aqueous non lipidic phase comprising the FVII can be assimilated to FVII-Tg-enriched lactoserum. This preparation is stored at −30° C. before continuing the process.

The total yield of the FVII recovery in this step is very satisfactory: 90% (91% extraction with phosphate +99% dialysis/concentration).

The non lipidic aqueous phase containing the FVII resulting from this step is perfectly clear and compatible with the further chromatographic steps.

At this stage, about 93 000 IU of FVII-Tg are extracted. The purity of FVII in this preparation is of the order 0.2%.

b) Purification of FVII

### 1. Chromatography on Hydroxyapatite Gel (Affinity Chromatography)

An Amicon 90 (diameter 9 cm—cross-section 64 cm²) column is filled with BioRad Ceramic Hydroxyapatite gel type I (CHT-I).

The gel is equilibrated with an aqueous buffer A consisting of a mixture of 0.025 M sodium phosphate and 0.04 M sodium chloride, pH 8.0. The whole preparation, stored at −30° C., is thawed in a water-bath, at 37° C., until the complete dissolution of the block of ice, then is injected onto the gel (linear flow rate 100 cm/h, that is 105 ml/min). The not retained fraction is discarded by passage of a buffer consisting of 0.25 M sodium phosphate and 0.04 M sodium chloride, pH 8.2, until return to baseline (RBL).

The elution of the fraction containing the FVII-Tg is carried out with the buffer B consisting of 0.025 M sodium phosphate and 0.4 M sodium chloride, pH 8.0. The eluted fraction is collected until return to baseline. The compounds are detected by absorbance measurements at λ=280 nm.

This chromatography allows to recover more than 90% of FVII-Tg, while removing more than 95% of lactic proteins. The specific activity (S.A.) is multiplied by 25. At this stage, about 85 000 IU of FVII-Tg with a purity of 4% are available.

### 2. 100 kDa Tangential Filtration and 50 kDa Concentration/Dialysis

The whole of the eluate from the previous step is filtered in tangential mode through a 100 kDa ultrafiltration membrane (Pall OMEGA SC 100K-0.1 m²). The FVII is filtered through the 100 kDa membrane, while proteins with a molecular weight higher than 100 kDa are not filterable.

The filtered fraction is further concentrated to a volume of about 500 ml, then dialysed on a 50 kDa ultrafilter described hereabove. The dialysis buffer is 0.15 M sodium chloride.

At this stage of the process, the product is stored at −30° C. before passage in ion exchange chromatography.

This stage allowed to reduce the charge of proteins with a molecular weight higher than 100 kDa and in particular the pro-enzymes. The treatment on the 100 kDa membrane allows to retain about 50% of proteins, among which the high molecular weight proteins, while 95% of the FVII-Tg, that is 82 000 IU of FVII-Tg are filtered.

This treatment allows to reduce the risk of proteolytic hydrolysis in the further steps.

3. Chromatographies on Q-Sepharose® FF Gel (Step d)—Process A)

These three successive chromatographies on ion exchange gel Q-Sepharose® Fast Flow (QSFF) are carried out in order to purify the active ingredient, to allow the activation of FVII to activated FVII (FVIIa) and finally to concentrate and to formulate the composition of FVII. The compounds are detected by absorbance measurements at λ=280 nm.

### 3.1 Q-Sepharose® FF 1 Step—Elution <<High Calcium>>

A 2.6 cm diameter (cross-section 5.3 cm²) column is filled with 100 ml of Q-Sepharose® FF (GE Healthcare) gel.

The gel is equilibrated with 0.05 M Tris, pH 7.5.

The whole fraction stored at −30° C. is thawed in a water bath, at 37° C., until the complete dissolution of the ice bloc. The fraction is diluted to ½[v/v] with the equilibrating buffer prior to the injection into the gel (flow rate 13 ml/min, that is a linear flow rate of 150 cm/h) then the not retained fraction is discarded by passage of the buffer until RBL.

A first protein fraction with a low content of FVII is eluted at 9 ml/min (that is 100 cm/h) with a buffer of 0.05 M Tris and 0.15 M sodium chloride, pH 7.5, and is subsequently discarded. A second FVII-rich protein fraction is eluted at 9 ml/min (that is 100 cm/h) with a 0.05 M Tris and 0.05 M sodium chloride and 0.05 M calcium chloride buffer, pH 7.5.

This second fraction is dialyzed on a 50 kDa ultrafilter already described hereabove. The dialysis buffer is 0.15 M sodium chloride. This fraction is stored at +4° C. overnight, prior to the second ion exchange chromatography passage.

This step allows to recover 73% of FVII (that is 60000 IU of FVII-Tg), while eliminating 80% of the accompanying proteins. This allows also the activation of FVII to FVIIa.

### 3.2 Q-Sepharose® FF 2 Step—Elution <<Low Calcium>>

A 2.5 cm diameter (cross section 4.9 cm²) column is filled with 30 ml of Q-Sepharose® FF (GE Healthcare) gel.

The gel is equilibrated with a buffer 0.05 M Tris, pH 7.5.

The previous eluted fraction (second fraction), stored at +4° C., is diluted prior to the injection onto the gel (flow rate 9 ml/min, that is a linear flow rate of 100 cm/h).

After the injection of the second fraction, the gel is washed with the equilibrating buffer for the removal of the not-retained fraction, until the RBL.

A fraction containing a very high purity FVII is eluted at 4.5 ml/min (that is 50 cm/h) with 0.05 M Tris, 0.05 M sodium chloride and 0.005 M calcium chloride, pH 7.5.

About 23 000 IU of FVII-Tg were purified, that is 12 mg of FVII-Tg.

This step allows to remove more than 95% of the associated proteins (female rabbit milk proteins).

This eluate, with a purity higher than 90%, exhibits structural and functional features near to the natural molecules of human FVII. The eluate is concentrated and formulated by a third ion exchange chromatography.

### 3.3 Q-Sepharose® FF 3 Step—Elution <<Sodium>>

A 2.5 cm diameter (cross section 4.9 cm²) column is filled with 10 ml of Q-Sepharose® FF (GE Healthcare) gel.

The gel is equilibrated with a buffer 0.05 M Tris, pH 7.5.

After the injection of the fraction, the gel is washed with the equilibrating buffer for the removal of the not-retained fraction, until the RBL.

The eluted, purified, fraction from the previous step is diluted five times with purified water for injection (PWI) prior to the injection into the gel (flow rate 4.5 ml/min, that is a linear flow rate 50 cm/h).

Afterwards, the FVII-Tg is eluted with a flow rate of 3 ml/min (that is 36 cm/h) with the buffer 0.02 M Tris and 0.28 M sodium chloride, pH 7.0.

A composition of FVII-Tg was prepared in form of a concentrate with a purity higher than 95%. The product is compatible with an intravenous injection. The process gives a cumulated yield of 22%, thus allowing to purify at least 20 mg of FVII per litre of treated milk.

The Table A resumes the process steps according to a preferred embodiment of the invention for providing the composition of purified FVII, and provides different yields, purities and specific activities obtained in each step.

Afterwards, the FVII-Tg of the composition is subjected to different structural analyses, such as described in the following examples.

### Example 3

### Characterization of the Glycosylation Sites and of the Glycopeptides by MS-ESI

The N-glycosylation sites of FVII-Tg, of FVIIa,p (plasma FVII) and of FVIIa,r were identified by LC-ESIMS(/MS), confirmed by MALDI-TOFMS, and the relative proportions of the different glycans present on each site were determined by LC-ESIMS.

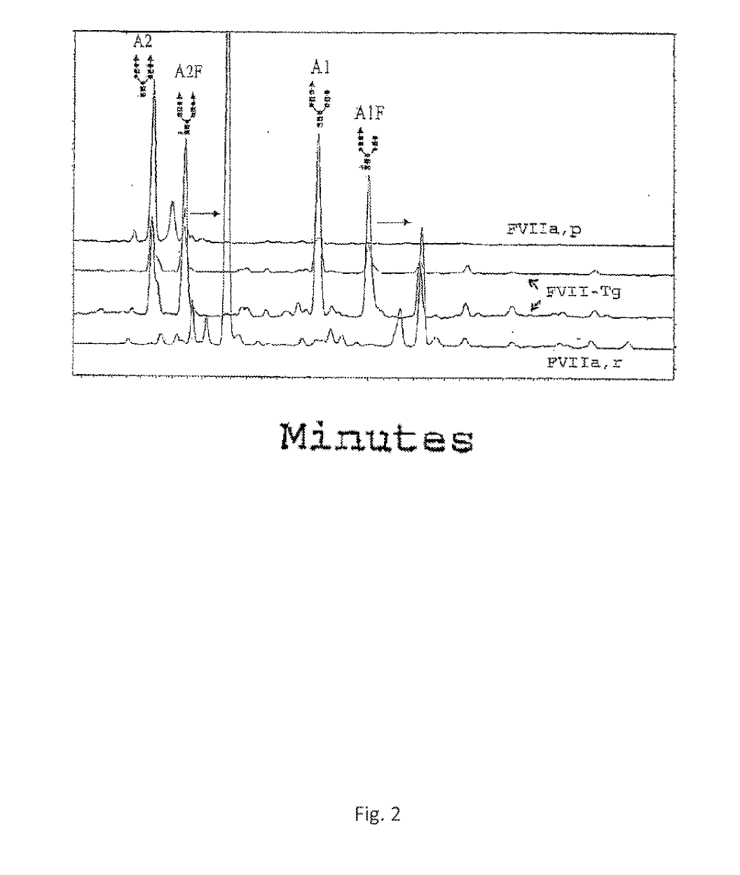

The FIG. 2 depicts the deconvoluted ESI spectra of glycopeptides containing both Asn glycosylated residues. The localisation of the glycosylation sites was confirmed by MALDI-TOF(/TOF) and by Edman's sequencing.

The analysis of mass spectra of the glycopeptides [D₁₂₃-R₁₅₂] and [K_{31.6}-R₃₅₃] of FVIIa,p, exhibiting the N-glycosylation sites Asn₁₄₅ and Asn₃₂₂, respectively, reveals the presence of a biantennary, bisialylated non fucosylated (A2) (observed mass of the glycopeptide containing Asn₁₄₅: 5563.8 Da) and a fucosylated form (A2F) (observed mass of the glycopeptide with Asn₁₄₅: 5709.8 Da). Also noted for Asn₁₄₅ the presence of triantennary, trisialylated, non fucosylated (A3) (observed mass 6220.0 Da) and fucosylated (A3F) (observed mass 6366.1 Da).

For the FVIIa,r, Asn₁₄₅ is modified by glycans of A2F, A1F type and “A1F”, this one corresponding to monosialylated form with a GalNAc terminal position on the other antenna. The presence of glycans A3F (triantennary, trisialylated, fucosylated forms) is noted.

For the FVII-Tg, the analysis of mass spectra of glycopeptides [D₁₂₃-R₁₅₂] and [K_{31.6}-R₃₅₃] of the FVII-Tg, presenting the N-glycosylation sites Asn₁₄₅ and Asn₃₂₂, respectively, reveals the presence of biantennary, bisialylated, non fucosylated (A2) forms (observed mass of the glycopeptide containing Asn₁₄₅: 5563.8 Da) and fucosylated forms (A2F) (observed mass: 5709.7 Da). The presence of majority oligosaccharides, located on Asn₁₄₅, biantennary, monosialylated and non fucosylated (A1) (observed mass: 5272.3 Da) and fucosylated (A1F) (observed mass: 5418.7 Da). The triantennary forms are poorly represented. It should be noted that no monosialylated form with a GalNAc in terminal position on the other antenna is present.

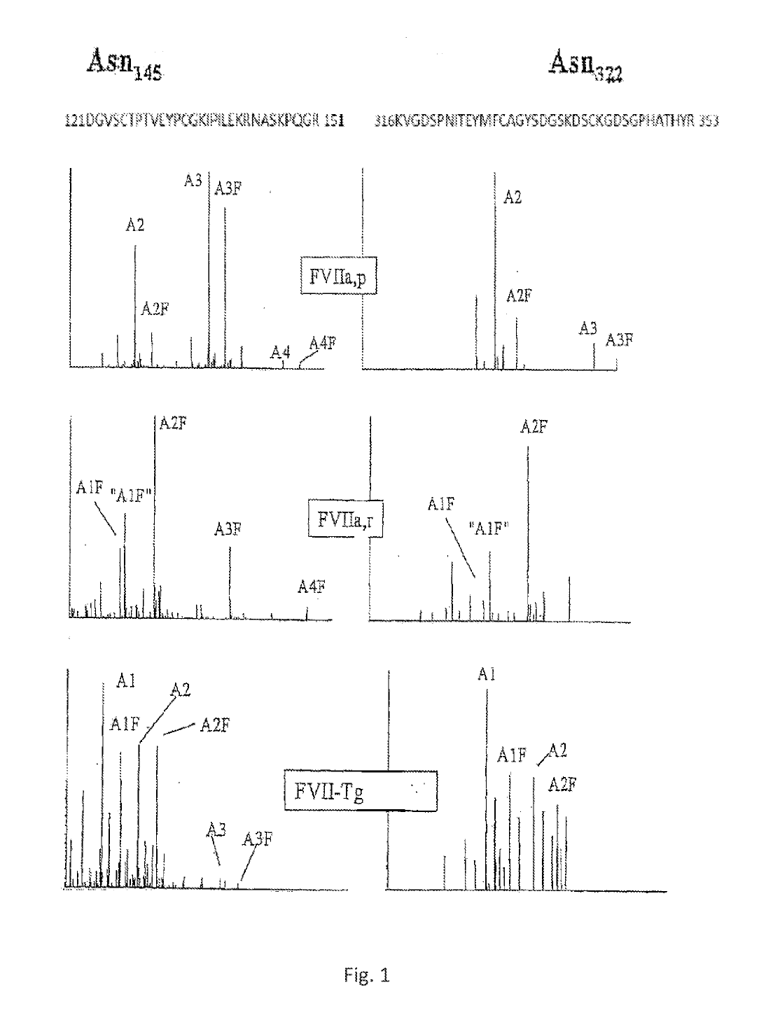

Relating to the majority glycoforms of Asn₃₂₂, the same glycan structures are observed in different proportions. The FIG. 1 shows the presence of less mature forms (less antennary and sialylated) as on the Asn₁₄₅. For example, the triantennary forms are less represented on Asn₃₂₂ by comparison with Asn₁₄₅ for the plasma product and are absent on the FVIIa,r and FVII-Tg. It should also be noted that the Asn 145 and 322 are glycosylated to 100%. Although solely semi-quantitative, these results are in agreement with the quantitative data obtained by HPCE-LIF and NP-HPLC.

### Example 4

### Quantification of N-Glycans by HPCE-LIF

The identification and quantification of N-linked oligosaccharides are carried out by HPCE-LIF after deglycosylation by PNGase F. Samples of FVII are treated with exoglycosidases (sialidase(ratio ENZYME/SUBSTRATE 1 mlU/10 μg), galactosidase, hexnacase (kit Prozyme), fucosidase (ratio E/S: 1 mUl/10 μg) in a way to ensure the identification and quantification of each isolated structure. The obtained glycans are labelled with a fluorochrome and separated depending on their mass and their charge. Two standards (homopolymers of glucose, oligosaccharidic) allow to identify the structures. The quantification is performed by integration of each peak reduced, in percentage, to the whole of quantified oligosacharides.

A capillary electrophoresis apparatus ProteomeLab PA800 (Beckman Coulter) is used, the capillary of which is N—CHO <<coated>> (Beckman-Coulter) of 50 cm×50 μm internal diameter. A separation buffer <<gel buffer-N>> (Beckman-Coulter) is used. The migration is performed by applying a voltage of 25 kV, for 20 min, at 20° C. The detection is performed by a laser at λ_{excitation} 488 nm and λₑₘᵢₛₛᵢₒₙ 520 nm.

The proportion of fucosylation is calculated, after deglycosylation at the same time with sialidase, galactosidase and hexnacase, by the relation between the surfaces of the peaks corresponding to the “core” and the fucosylated “core”.

The glycans of FVIIa,p are in majority of biantennary, bisialylated, non fucosylated (A2) type, and of biantennary, bisialylated, fucosylated (A2F) type. The glycan profiles of FVII-Tg reveal the presence of biantennary, monosialylated, fucosylated or non fucosylated (A1F, A1), and of biantennary, bisialylated, fucosylated or non fucosylated (A2F, A2) forms. The distribution varies between these different forms in both charges.

The FVIIa,r exhibits biantennary, sialylated, fucosylated glycan forms with a majority of A2F forms, and biantennary, monosialylated, fucosylated (A1F) forms. Atypic migration times are observed for the A2F and A1F forms compared to migration times usually encountered with these structures.

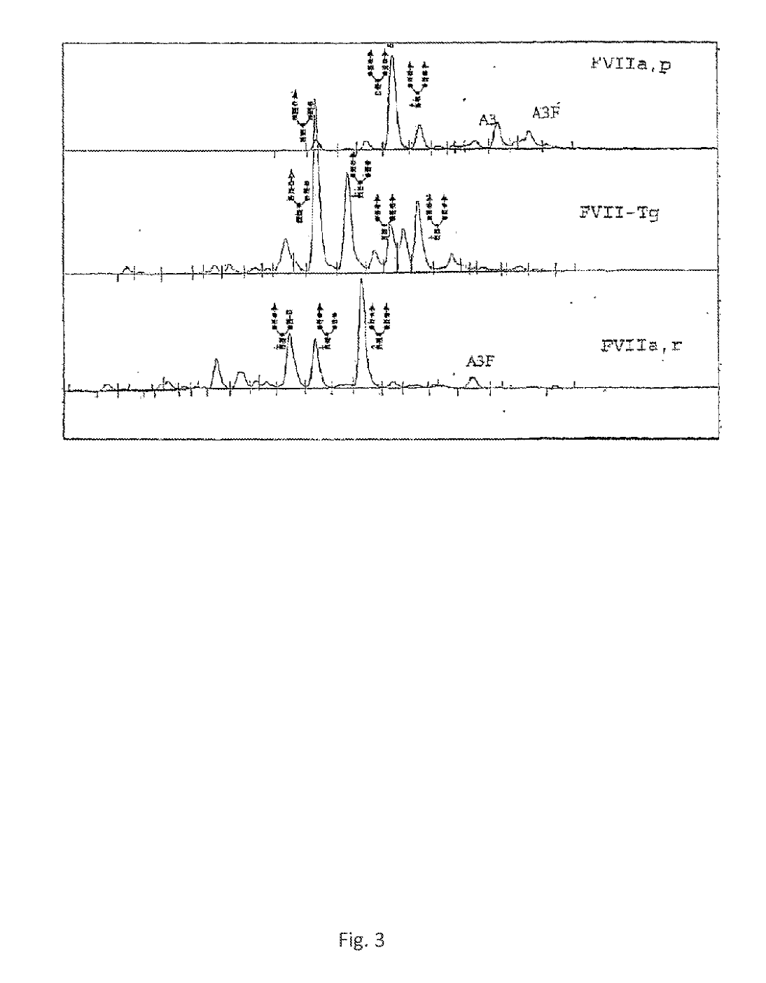

The glycan profiles of both batches (A and B) of FVII-Tg (cf FIG. 3—both electropherograms in center) reveal the presence of biantennary, monosialylated, fucosylated or non fucosylated (A1F, A1), and biantennary, bisialylated, fucosylated or non fucosylated (A2F, A2) forms.

TABLE 1
Summary of percentage of sialylated forms resulting
from native samples of different batches of FVII.

| | | FVII-Tg | FVII-Tg | |
|---|---|---|---|---|
| Percentage | FVIIa,p | batch A | batch B | FVIIa,r |
| Native A2 | 41.9 | 19.3 | 13.9 | — |
| A2F | 8.9 | 14.8 | 21.5 | 44.8 |
| A1 | 2.6 | 38.4 | 25.2 | — |
| A1F | — | 11.7 | 22.2 | 16.5 |
| Total A2 + A2F | 50.8 | 34.1 | 35.4 | 44.8 |
| Total A1 + A1F | 2.6 | 50.1 | 47.4 | 16.5 |

The quantitative analysis of different glycan forms (Table 1) shows that, for the FVIIa,p, the predominance of sialylated forms with 51% of bisialylated glycans (A2 and A2F), and 30% of triantennary sialylated non fucosylated and fucosylated (G3 and G3F respectively) forms (results not shown). The FVII-Tg (batches A and B) is less sialylated than the FVIIa,p with 35% biantennary, bisialylated forms, and only 6% of triantennary, sialylated forms (results not shown). The main forms are monosialylated with 50% of structures A1 and A1F. Also the FVIIa,r is less sialylated than the FVIIa,p with 45% of A2F structures and only 6% of triantennary, sialylated glycans (results not shown). The lack of non fucosylated forms of FVIIa,r is noted.

TABLE 2
The proportion of fucosylation of different FVII

| | | FVII-Tg | FVII-Tg | |
|---|---|---|---|---|
| | FVIIa,p | batch A | batch B | FVIIa,r |
| Proportion of | 16.2 | 23.6 | 41.8 | 100 |
| fucosylation (%) | | | | |

The results show a low proportion of fucosylation of the FVIIa,p (16%), a proportion of fucosylation from of 24 to 42% of the FVII-Tg and a 100% fucosylation of the FVIIa,r.

### Example 5

### Quantification of N-glycans by NP-HPLC

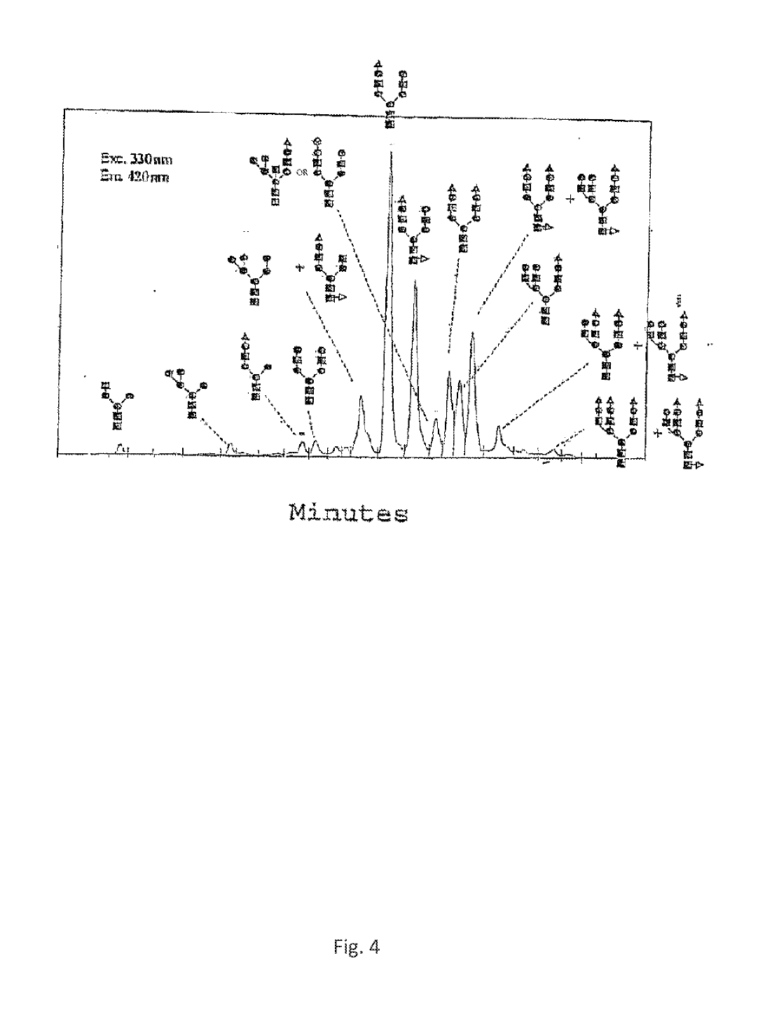

The qualitative and quantitative analysis of the N-glycosylation of FVIIa,p, FVIIa,r and FVII-Tg was studied by NP-HPLC (cf. FIG. 4). After desalting and drying of the protein, this protein is denaturated and reduced by methods known to persons skilled in the art. Afterwards, the glycans are released in an enzymatic reaction (endoglycosidase PNGase F), and purified by precipitation with ethanol. The thus obtained glycans are labelled with a fluorophore, 2-aminobenzamide (2-AB). The labelled glycans are separated, depending on their hydrophilic character, by normal phase HPLC chromatography on a Amide-80 column, 4.6×250 mm (Tosohaas) thermostatised at 30° C.

Prior to the injection of the sample, the column is equilibrated with a buffer to 80% of acetonitril. The oligosaccharides are eluted in an increasing gradient of 50 mM ammonium formate, pH 4.45, for periods of time higher than or equal to 140 minutes. The detection is carried out by fluorimetry at λ_{excitation} at 330nm and λₑₘᵢₛₛᵢₒₙ at 420 nm.

The chromatographic profile of FVIIa,p shows that the majority glycans are of biantennary, bisialylated (A2) type with a proportion of 39%. Also are observed, in lesser amounts, biantennary, bisialylated, fucosylated (A2F), monosialylated (A1) and trisialylated fucosylated and non fucosylated (A3F and A3) forms.

The NP-HPLC analysis carried out on the FVII-Tg confirms the presence of oligosaccharides in majority of type A1, at a proportion of 27%. The A1F, A2 and A2F forms are less represented and the triantennary forms are present in traces. This reveals a difference of sialylation between FVIIa,p and the Factor FVII-Tg (batch B), less sialylated.

The same analysis, carried out on a Factor FVIIa,r, reveals the majority forms of type A2F present in an amount of 30%. The forms A1F are less represented and the triantennary forms are present in traces. The analysis of FVIIa,r also shows an important time lag of the retention time of forms A1F and A2F suggesting forms different from those present in the FVIIa,p and in the FVII-Tg.

The set of these results is consistent with those obtained by HPCE-LIF.

### Example 6

### Identification by MALDI-TOFMS

The mass spectrometry MALDI-TOF MS (Matrix-Assisted Laser Desorption/Ionisation Time of Flight Mass Spectrometry) is a technique measuring the molecular weight of peptides, proteins, glycans, oligonucleotides, and the majority of ionisable polymers with a high exactitude.

The peptides, proteins and glycans to be analysed are mixed with a matrix which absorbs at a wavelength of the employed laser. The main matrices are -cyano-4-hydroxycinnamic acid (HCCA) for peptides, sinapinic acid (SA) for proteins and 2,5-dihydroxybenzoic acid (DHB) for oligosaccharides analysis.

The method consists of an irradiation of the co-crystals matrix/analyte with a pulsed laser, this induces the joint desorption of the matrix and the analyte molecules. After ionisation in gaseous phase, the analyte molecules pass through the detector in the flight time. As the masses and the flight times are directly related, the measuring of the latter allows to determine the mass of the target analyte. The identification is carried out by measuring of the observed mass, by comparing to the theoretical mass. The sequencing can be carried out in MS/MS mode based on the obtained fragment ions. The employed instrument is a Bruker Autoflex 2 operating in TOF and TOF/TOF modes.

In order to identify the glycan forms present in the FVII-Tg and the FVIIa,r, MALDI-TOF MS analyses were carried out on elution fractions resulting from preparative NP-HPLC.

The MALDI-TOF analysis of the FVII-Tg allowed to confirm the identification of glycans separated by NP-HPLC, namely the majority monosialylated A1 forms and the minority forms of A1F, A2F and A2 type.

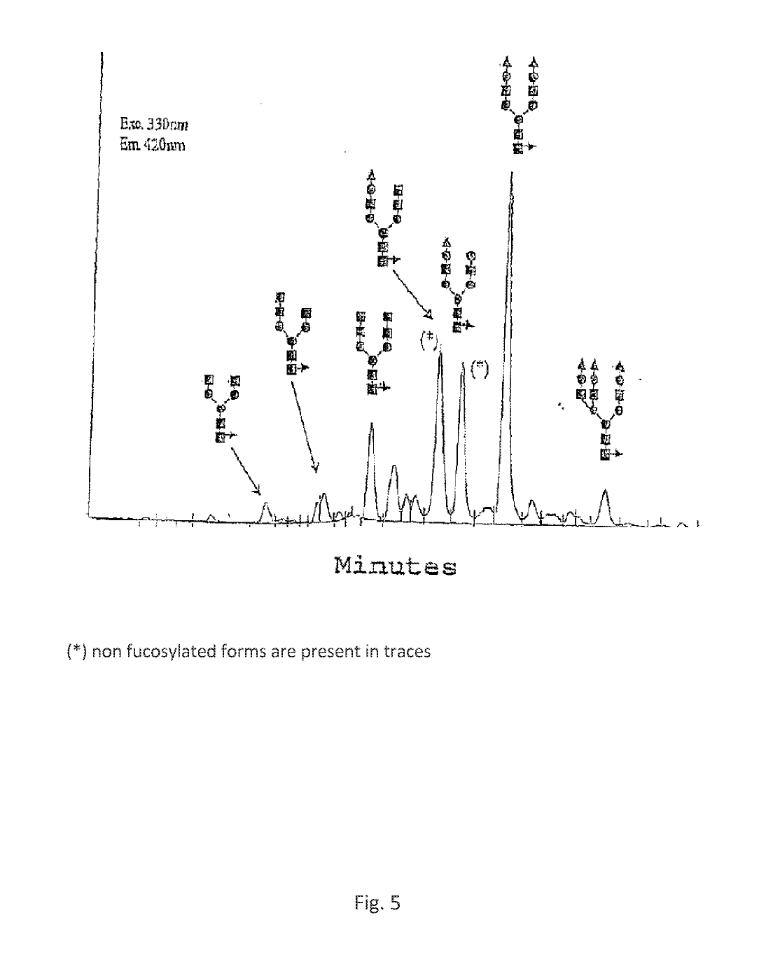

This study also allowed to identify the minority forms of triantennary bisialylated and trisialylated type, hybride forms and oligomannoses of Man5 and Man6-P-HexNAc type (cf. FIG. 5).

The MALDI-TOF MS analysis carried out on the FVIIa,r revealed the presence of glycan forms showed on the FIG. 6. The Factor FVIIa,r is nearly completely fucosylated, unlike the FVII-Tg which is only partially fucosylated. It is noted that the majority glycan form is A2F with a quantified proportion by NP-HPLC to 30%. The biantennary, monosialylated, fucosylated forms (A1F), and comprising a GalNAc in terminal position on the other antenna, are identified and the neutral biantennary fucosylated forms, as well, with the Hex-NAc-HexNAc moieties on one and/or two antennae. The presence of glycans of triantennary, trisialylated and fucosylated forms is also noted. The non fucosylated forms are present in traces.

### Example 7

### HPCE-LIF Analysis of the Sialic Acids—Galactose Link

Concerning the study of the sialic acid-galactose link (“branching”), the exprimental procedure is similar to that set forth in Example 4. After deglycosylation with PNGaseF, the oligosaccharides are treated with specific exosialidases in a way to ensure the identification of the link and the quantification of each isolated structure. The employed sialidases are recombinant enzymes obtained from S. pneumoniae (α2-3 link specific, 0.02 IU, E/S=0.4 m/m), C. perfringens (α2-3- and α2-6-links specific, 0.04 IU, E/S=0.1 m/m) and A. urefaciens (hydrolysing the α2-3, α2-6, α2-8 and α2-9 links, 0.01 IU, E/S=0.05 m/m).

The analyses have shown that the FVIIa,r has biantennary, sialylated, fucosylated glycan forms with the majority A2F, and biantennary, monosialylated, fucosylated (A1F) forms. Atypical migration times are observed for these A2F and A1F structures compared with the migration times usually encountered with these forms. Especially, these oligosaccharidic sialylated forms exhibit atypical migration times in HPCE-LIF and NP-HPLC compared to those of the FVII-Tg. On the other hand, no particular sialic acid, other than Neu5Ac, was revealed in the analysis of the composition of monosaccharides and the mass spectrometry means reveal glycans with a mass according to bisialylated types. Finally, the desialylation of glycans of FVIIa,r allows to find chromatographic and electrophoretic behaviors equivalent to those of oligosaccharides of the FVII-Tg.

These differences in the chromatographic and electrophoretic behaviour can therefore be explained on the basis of a different branching of sialic acids. This assumption was assessed by different approaches by HPCE-LIF and MS.

The results are resumed in Table 3 herebelow.

TABLE 3
Branchings of sialic acids on the different batches of FVII.

| | Sialylation (%) | α2-3 (%) | α2-6 (%) | α2-8 (%) |
|---|---|---|---|---|
| FVIIa,r | 91 | 100 | 0 | 0 |
| FVII-Tg | 96 | 0 | 100 | 0 |
| batch C | | | | |

The results show an isomery at the sialic acids level distinct between both FVII. Indeed, the sialic acids of FVIIa,r imply α2-3-links, while the FVII-Tg exhibits α2-6 branchings. The differences in the HPCE-LIF and NP-HPLC behaviours noted for the glycans of FVIIa,r compared to FVII-Tg are related to these differences in isomery at sialic acids level.

### Example 8

### In Vitro Resialylation of the FVII-Tg

The literature (Zhang X. et al, Biochim. Biophys. Acta 1998, 1425; 441-52) mentions that a more complete sialylation of a glycoprotein contributes to the improved stabilities in vitro and in vivo. The aim of this study is to demonstrate the feasibility of a sialylation in vitro.

The resialylation was carried out by use of a α2,6-(N)-sialyltransferase (rat, Spodotera frugiperda, S.A.≧1 unit/mg (S.A.: Specific Activity), 41 kDa, Calbiochem) and of the substrate cytidine-5′-monophospho-N-acetylneuraminic acid (Calbiochem). These two reagents are stored at −80° C. due to their instability. The sialylation substrate (cytidine-5′-monospho-N-acetylneuraminic acid) and the enzyme α2,6-(N)-sialyltransferase) are mixed in the reaction buffer, over night at 37° C. The employed reaction buffer is 50 mM of morpholino-3 propanesulfonic acid, 0.1% Tween®80, 0.1 mg/ml BSA (bovine serum albumine), adjusted to a pH 7.4 (reagents Sigma).

The Table 4 herebelow resumes the experimental conditions.

TABLE 4
Summary of the experimental conditions

| | Control | Resialylated |
|---|---|---|
| | native FVII | FVII |
| FVII (μg) | 50 | 50 |
| Reaction buffer (μl) | 200 | 200 |
| CMP-Neu5Ac (μl) | — | 2 |
| (cytidine-5′-monophospho-N- | | |
| acetylneuraminic acid) | | |
| A2,6-NST (μl) | 20 | 20 |
| (α2,6-(N)-sialyltransferase) | | |
| Incubation | Overnight | Overnight |

The electropherogram of the native FVII-Tg, such as obtained after purification of Example 2 (FIG. 7, bottom profile), shows the majority biantennary, monosialylated A1 form (42%) and the less represented structures A2, A2F and A1F. After resialylation (FIG. 7, top profile) the monosialylated form represents only 6% to the benefit of the bisialylated form, especially non fucosylated, turning highly majority (52%).

The quantification of glycans before and after the resialylation is shown in the Table 5 herebelow.

TABLE 5
Quantification of oligosaccharidic structures
before and after sialylation

| | Native FVII-Tg | Resialylated FVII-Tg |
|---|---|---|
| A2 | 19.8 | 52.4 |
| A2F | 15.5 | 25.1 |
| A1 | 42.1 | 6.4 |
| A1F | 13.6 | 11.6 |
| Neutral | 9.0 | 4.5 |
| % Sialylation | 91.1 | 95.5 |
| Proportion of bisialylated | 35.3 | 77.5 |

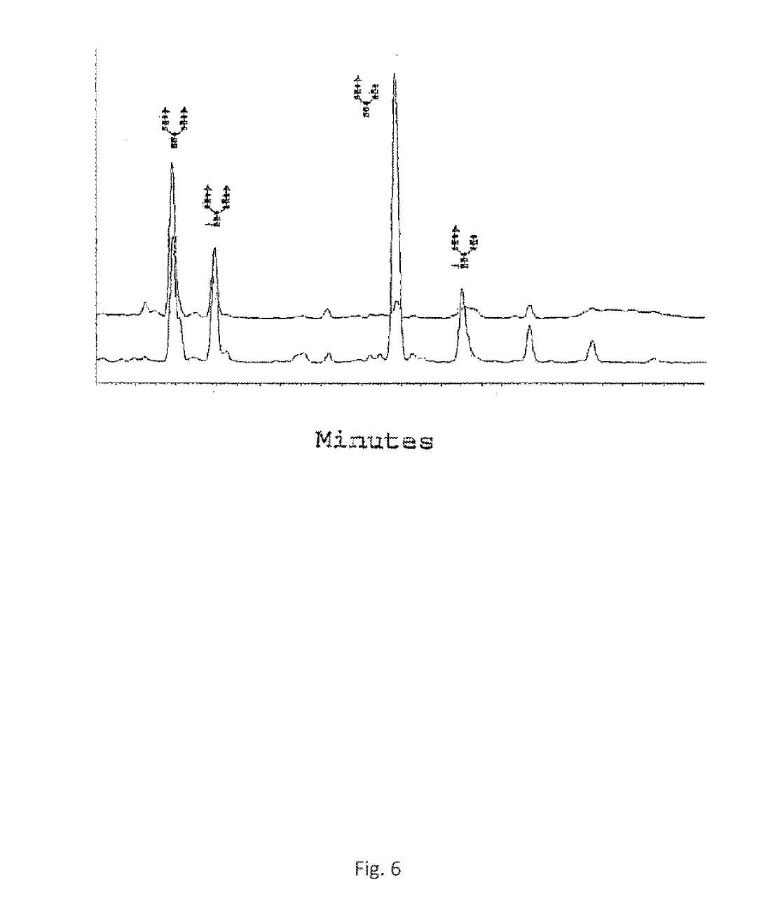

The kinetics of sialylation of the transgenic FVII is depicted in the FIG. 8. This study shows the efficiency of resialylation in vitro with a proportion of bisialylated forms increased by more than 100%.

### Example 9

### Comparative Pharmacokinetic STUDY on Rabbit of a Transgenic Non Resialylated FVII (FVII Tg NRS) Compared to a Transgenic Resialylated FVII (FVII Tg RS), Resulting From the Example 8)

The aim of this study is the comparison of pharmacokinetic profiles of the FVII-TgRS with the FVII-TgNRS on a New Zealand male vigil rabbit.

The tested dose is 200 μg/kg per animal, what is the double of the therapeutic dose of recombinant FVII administered to humans.

The blood takings are done on J-4 (4 days before the injection of the product) and on J1 (day of the injection of the product) at T0.17 h (day of the injection, 10 min. after the injection), T0.33 h (day of the injection, 20 min. after the injection), T1 h (day of the injection, 1 hour after the injection), T3 h (day of the injection, 3 hours after the injection), T6 h (day of the injection, 6 hours after the injection), T8 h (day of the injection, 8 hours after the injection).

The dosage of FVII:Ag (antigen of FVII) are performed with an ELISA (Asserachrom kit). The results of dosages of the FVII:Ag dosage in rabbit plasma allow to determine, on one hand, the removal profiles and, on the other hand, the pharmacokinetic parameters. The posologies and the experimental groups are shown in the Table 6.

TABLE 6
Posologies and experimental groups

| Experimental | Administered | Animals | Dose at | Proportion of | Injected |
|---|---|---|---|---|---|
| group | product | number/weight | J1 | protein/FVII:Ag | volume |
| Group 1 | FVII-Tg RS | 3 rabbits (1 to 3) | 200 μg/kg | 143 μg/mL proteins | 1.4 mL/kg |
| | | 2.315 ± 0.124 kg | | FVII:Ag = 253.7 ± 5.8 U/ml | |
| Group 2 | FVII-Tg NRS | 3 rabbits (4 to 6) | 200 μg/kg | 145 μg/mL proteins | 1.4 mL/kg |
| | | 2.352 ± 0.130 kg | | FVII:Ag = 263.7 ± 2.9 U/ml | |
| Group 3 | NaCl 0.9% | 3 rabbits (7 to 9) | NA | NA | 1.4 mL/kg |

NA: Not Applicable

The removal curves are depicted on the FIG. 9.

The results are reproduced in the Table 7.

TABLE 7
Results

| | Dose | T½ | MRT | Cmax | Recovery | AUC | Cl | Vd |
|---|---|---|---|---|---|---|---|---|
| Parameters PK | (U) | (h) | (h) | (mU/ml) | (%) | (h × mU/ml) | (ml/h) | (ml) |
| FVII-TgRS | 822 ± 44 | 1.85 ± 0.08 | 2.93 ± 0.09 | 2060 ± 394 | 20 ± 4 | 2563 ± 335 | 320 ± 46 | 856 ± 151 |
| FVII-Tg NRS | 868 ± 48 | 1.76 ± 0.08 | 2.81 ± 0.03 | 1797 ± 389 | 17 ± 4 | 1863 ± 346 | 479 ± 103 | 1216 ± 288 |

With the administered doses, the removal half-life, the mean residence time (MRT), the maximal concentration (Cmax) and the proportion of recovery (<<recovery>>) are comparable in both experimental groups.

The FVII-TgRS exhibits a different kinetics profile than the FVII-TgNRS. The resialylation of the FVII-Tg improves in a unnoticeable way the half-life, the mean residence time (MRT), the Cmax and the <<recovery>>.

A difference is observed at the AUC parameters level (peak area), CI (clearance) and distribution volume (Vd) (This volume is obtained by dividing the administered or absorbed dose by the plasma concentration) suggesting a less important elimination of FVII-TgRS from the blood circulation.

The resialylation of the FVII-Tg induces an increase in the biodisponibility of the product by about 30%.

TABLE A

| | Volume | Amount of | Amount | Yield | Yield | SA | Purity |
|---|---|---|---|---|---|---|---|
| Batch N°479030 | (ml) | proteins (mg) | FVII: Ag (U) | FVII/step (%) | FVII/cumulated (%) | (U/mg) | FVII (%) |
| Pool of raw milk | 500 | 42750 | 103450 | 100% | 100% | 2.4 | 0.12% |
| Phosphate clarification | 4785 | ND | 93650 | 91% | 91% | — | — |
| Concentration/dialysis (UF 50 kDa) | 657 | 29610 | 93233 | 99% | 90% | 3.1 | 0.20% |
| Hydroxyapatite eluate (CHT-I) | 2644 | 1071 | 85692 | 92% | 79% | 80.0 | 4.0% |
| Tangential filtration (UF 100 kDa) | 459 | 518 | 81684 | 95% | 72% | 157.6 | 7.9% |
| QSFF1 eluate (High Ca++) | 402 | 105 | 59757 | 73% | 58% | 572 | 28.6% |
| QSFF2 eluate (Low Ca++) | 157 | 12.8 | 22447 | 38% | 22% | 1749 | 87% |
| QSFF3 eluate (Sodium) | 42.5 | 12.7 | 21929 | 98% | 21% | 1727 | 86% |
| Finished product (sterilization 0.2 μm) | 50 | 12.4 | 23197 | 106% | 22% | 1878 | 94% |

1. A composition of transgenic Factor VII (FVII), each molecule of Factor VII of the composition containing glycan forms bound to N-glycosylation sites, and wherein more than 50% of the glycan forms bound to N-glycosylation sites of Factor VII are biantennary, bisialylated and non fucosylated glycan forms and wherein all sialic acids of Factor VII of said composition imply α2-6-links.

2. The composition according to claim 1, wherein in the proportion of biantennary, bisialylated, fucosylated and non fucosylated forms is higher than 50%.

3. The composition according to claim 1, wherein among all the molecules of Factor VII of said composition, the proportion of fucose is comprised between 20% and 50%.

4. The composition according to claim 2, wherein among all the molecules of Factor VII of said composition, the proportion of fucose is comprised between 20% and 50%.

5. The composition according to claim 1, characterized in that said FVII is activated.

6. The composition according to claim 1, for use as a medicament.

7. The use of the composition recited in any one of claims 1 to 5, for preparing a medicament for the treatment of patients suffering from haemophilia.

8. The use of the composition recited in any one of claims 1 to 5, for preparing a medicament for the treatment of multiple hemorragic traumas.

9. The use of the composition recited in any one of claims 1 to 5, for preparing a medicament for the treatment of bleedings due to an overdose of anticoagulants.

10. A pharmaceutical composition comprising composition recited in any one of claims 1 to 5, and an excipient and/or a pharmaceutically acceptable carrier.


SEQUENCE LISTING (1 sequence)

SEQ ID NO: 1 - protein, 406 aa, Homo sapiens [sequence omitted]